(19) United States
(12) Patent Application Publication
(10) Pub. No.: US 2008/0199455 A1
(43) Pub. Date: Aug. 21, 2008

(54) Conformation specific antibodies that bind trefoil factors and methods of treating cancers and proliferation disorders using same

(76) Inventor: Peter E. Lobie, Auckland (NZ)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/906,968

(22) Filed: Oct. 3, 2007

Related U.S. Application Data
(60) Provisional application No. 60/849,266, filed on Oct. 3, 2006.

(51) Int. Cl. A61K 39/00; C07K 16/00; G01N 33/53

(52) U.S. Cl. 424/130.1; 530/387.1; 530/387.3; 435/7.1

(57) ABSTRACT

The present invention relates to conformation specific antibodies to TFF and compositions thereof. The present invention also relates to methods of regulation of cellular proliferation and/or survival, particularly methods for the treatment of cancers, tumors and proliferative disorders.

### RELATED APPLICATIONS

This patent application claims the benefit of U.S. Provisional Patent Application Ser. No. 60/849,266, filed Oct. 3, 2006, which is hereby incorporated by reference in its entirety.

### FIELD OF THE INVENTION

The invention relates generally to conformation specific antibodies to TFF and methods of using such antibodies to modulate, treat, prevent or delay the progression of cancer or a proliferative disorder.

### BACKGROUND OF THE INVENTION

The regulation and control of proliferation and/or survival of cells in animals is a complex process involving a number of cellular factors and their interactions with one another. Mutations or alteration in expression in any number of these cellular factors can result in uncontrolled proliferation or growth of cells and ultimately lead to the development of tumors and cancer.

Hormones and/or growth factors are involved in the normal regulation and control of cellular growth and development. For example, growth hormone (GH) is involved in normal pubertal mammary gland development (Walden et al., Endocrinology 139, 659-662, 1998; Kleinberg, J. Mammary Gland Biol. Neoplasia. 2, 49-57, 1997; Bchini et al., (1991) Endocrinology 128, 539-546, 1991; Tornell et al., Int. J. Cancer 49, 114-11, 1991; Nagasawa et al., Eur. J. Cancer Clin. Oncol. 21, 1547-1551, 1985; Swanson and Unterman, Carcinogenesis 23, 977-982, 2002; Stavrou and Kleinberg, Endocrinol. Metab. Clin. North Am. 30, 545-563, 2001; Okada and Kopchick, Trends Mol. Med. 7, 126-132, 2001; Ng et al., Nat. Med. 3, 1141-1144, 1997) and expressed in normal human mammary gland (Raccurt et al., J. Endocrinol. 175, 307-318, 2002). Alterations in expression levels of hormones such as GH can result in aberrant proliferation of cells. For example, increased epithelial expression of the hGH gene is associated with the acquisition of pathological proliferation, and the highest level of hGH gene expression is observed in metastatic mammary carcinoma cells (Raccurt et al., J. Endocrinol. 175, 307-318, 2002). Such alterations in expression of autocrine hGH may result in the transformation of a normal cell to a cancer cell.

There is a need to understand further the effects of hormones and/or growth factors on the development of proliferative disorders, including identifying any cellular factors which promote cell proliferation, cell survival and/or oncogenic transformation. This will aid in the identification of means for the regulation of proliferation and/or survival, and particularly means for the treatment of proliferative disorders such as cancer.

### SUMMARY OF THE INVENTION

The invention provides conformation specific antibodies to TFF and methods of using such antibodies to modulate, e.g., reduce, inhibit, treat or prevent cancer or a proliferative disorder, and/or to modulate, e.g., reduce, inhibit, or delay the progression of a cancer or proliferative disorder.

The conformation specific antibodies to TFF described herein include antibodies that specifically bind a trefoil factor 1 (TFF1) polypeptide, wherein the antibody binds to a conformational epitope on the TFF1 polypeptide. For example, the conformational epitope is selected from a conformational epitope shown in Table 3.

The conformation specific antibodies to TFF described herein include antibodies that specifically bind a trefoil factor 1 (TFF1) polypeptide, wherein the antibody comprises an antigenic determinant selected from the antigenic determinants shown in Table 4.

The conformation specific antibodies to TFF described herein include antibodies that specifically bind a trefoil factor 1 (TFF1) homodimer polypeptide, wherein the antibody binds to a conformational epitope on the TFF1 polypeptide. For example, the conformational epitope is selected from a conformational epitope shown in Table 1.

The conformation specific antibodies to TFF described herein include antibodies that specifically bind a trefoil factor 1 (TFF1) homodimer polypeptide, wherein the antibody comprises an antigenic determinant shown in Table 2.

The conformation specific antibodies to TFF described herein include antibodies that specifically bind a trefoil factor 3 (TFF3) polypeptide, wherein the antibody binds to a conformational epitope on the TFF3 polypeptide. For example, the conformational epitope is selected from a conformational epitope shown in Table 5.

The conformation specific antibodies to TFF described herein include antibodies that specifically bind a trefoil factor 3 (TFF3) homodimer polypeptide, wherein the antibody binds to a conformational epitope on the TFF3 polypeptide. For example, the conformational epitope is selected from a conformational epitope shown in Table 5.

The conformation specific antibodies to TFF described herein include antibodies that specifically bind a trefoil factor 3 (TFF3) homodimer polypeptide, wherein the antibody comprises an antigenic determinant shown in Table 6.

The conformation specific antibodies to TFF described herein include chimeric antibody compositions that contain a TFF1 binding component and a TFF3 binding component.

The conformation specific antibodies to TFF described herein include antibodies produced by hybridoma cell lines and referred to herein as 1C6, 3F6, 2C5, 1D, 1A12, 3A2, 3A5, 3B8, 3F4, 3F12, 3G4, 1A11, 2B3, 3B4, 1C4, 2C12, 2A8, 3D7, 1E4, 2E2, 2H4, 1D8 and 2D7.

The conformation specific antibodies to TFF provided herein specifically bind to a trefoil factor polypeptide. For example, in some embodiments, the conformation specific antibodies to TFF bind to TFF1. In some embodiments, the conformation specific antibodies to TFF bind to TFF3. The invention also provides multivalent antibodies that recognize both TFF1 and TFF3. These antibodies are also referred to herein as multimeric antibodies. For example, in some embodiments, the TFF specific antibody is a heterodimer. In some embodiments, the TFF specific antibody is a chimeric antibody in which the antigen-binding fragment of the antibody from one species is fused with constant region from another species. For example, the TFF specific antibody is a humanized antibody.

The term “antibody” is used herein in the broadest sense and is intended to include intact monoclonal antibodies and polyclonal antibodies, as well as derivatives, variants, fragments and/or any other modification thereof so long as they exhibit the desired biological activity. Antibodies encompass immunoglobulin molecules and immunologically active portions of immunoglobulin (Ig) molecules, i.e., molecules that contain an antigen binding site that specifically binds (immunoreacts with) an antigen. These include, but are not limited to, polyclonal, monoclonal, chimeric, single chain, Fc, Fab, Fab′, and Fab₂ fragments, and a Fab expression library. Antibody molecules relate to any of the classes IgG, IgM, IgA, IgE, and IgD, which differ from one another by the nature of heavy chain present in the molecule. These include subclasses as well, such as IgG1, IgG2, and others. The light chain may be a kappa chain or a lambda chain. Reference herein to antibodies includes a reference to all classes, subclasses, and types. Also included are chimeric antibodies, for example, monoclonal antibodies or modifications thereof that are specific to more than one source, e.g., a mouse or human sequence. Further included are camelid antibodies or nanobodies. TFF binding antibodies also include multi-specific, e.g., bispecific, antibodies and functional fragments thereof. The terms “TFF binding antibodies” and “TFF antibodies” are used interchangeably herein. It will be understood that each reference to “antibodies” or any like term, herein includes intact antibodies, as well as any modifications thereof.

Conformation specific antibodies to TFF include those that bind to domains or residues that are exposed, e.g., outer loop structure residues in the tertiary structure of the protein in solution, participate in TFF dimerization, aggregation, as well as domains responsible for promoting cellular proliferation, survival, and oncogenicity. For example, the epitope binding specificity of the antibody includes a TFF sequence that contains a domain involved in stimulation of cell proliferation, survival and oncogenicity. For example, an antibody binds to a conformational epitope provided herein in Tables 1, 3, or 5. The antibody is a polyclonal antisera or monoclonal antibody or derivative of either of those. The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin.

TFF binding antibodies are used to modulate, e.g., reduce or otherwise inhibit, completely or partially, the ability of the target TFF molecule (i.e., the TFF antigen to which a given TFF binding antibody binds) to bind or otherwise interact with another TFF molecule. The additional TFF molecule can be the same as the target TFF molecule, as in the case of homo-multimerization, e.g., homodimerization, or the additional TFF molecule can differ from the target TFF molecule.

TFF binding antibodies are also used to modulate, e.g., reduce or otherwise inhibit, completely or partially, the ability of the target TFF molecule to bind or otherwise interact with a second molecule, such as, for example, a cognate TFF receptor molecule, an extracellular receptor or other cell-surface and/or intracellular signaling molecules.

Other TFF-binding antibodies are used to directly target TFF-over-expressing cells for destruction. In the latter case, the antibody, or fragment thereof, activates complement in a patient treated with the antibody. In some instances, the antibody mediates antibody-dependent cytotoxicity of tumor cells in the patient treated with the antibody. The antibody, or fragment thereof, is administered alone or conjugated to a cytotoxic agent. Binding of the antibody to a tumor cell expressing TFF results in impairment or death of the cell, thereby reducing tumor load. The antibody is optionally conjugated to a radiochemical, or a chemical tag which sensitizes the cell to which it is bound to radiation or laser-mediated killing.

Optionally, the antibody compositions contain a pharmaceutically acceptable carrier and/or a second compound. For example, the second compound is a chemotherapeutic or anti-neoplastic agent. Such agents are administered sequentially, e.g., prior to, or after the administration of the TFF antibody, or simultaneously, e.g., co-administration or co-therapy.

The conformation specific antibodies to TFF described herein are used in methods of inhibiting proliferation and/or survival of a tumor cell by contacting the cell, a biological sample suspected of containing a tumor cell, an extracellular receptor, such as a TFF receptor, or another cell-surface protein on the tumor cell with any of the conformation specific antibodies to TFF described herein, or with combinations of these antibodies.

The conformation specific antibodies to TFF described herein are used in methods of preventing cancer or a cell proliferation and/or survival disorder in a subject in need thereof by administering any of the conformation specific antibodies to TFF described herein, or by administering combinations of these antibodies.

The conformation specific antibodies to TFF described herein are used to treat a cancer or tumor. For example, the tumor or cancer is an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

The conformation specific antibodies to TFF described herein are used to treat a proliferative disorder. Proliferative disorders include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

The subject is a mammal, preferably a human suffering from or at risk of developing a tumor or cancer or proliferative disorder. The compositions and methods are also useful for veterinary use, e.g., in treating, cats, dogs, and other pets in addition to livestock, horses, cattle and the like.

The conformation specific antibodies to TFF described herein are also useful in a variety of diagnostic applications. The invention features a method for diagnosing cancer or a cell proliferation and/or survival disorder in a mammal by contacting a tissue or bodily fluid from the mammal with a conformational specific antibody to TFF under conditions sufficient to form an antigen-antibody complex and detecting the antigen-antibody complex. Cancers or tumors detected using the conformation specific antibodies to TFF described herein include an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor. Proliferative disorders detected using the conformation specific antibodies to TFF described herein include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

Patient derived tissue samples, e.g., biopsies of solid tumors, as well as bodily fluids such as a CNS-derived bodily fluid, blood, serum, urine, saliva, sputum, lung effusion, and ascites fluid, are contacted with a conformation specific antibodies to TFF.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1A is a photograph depicting the results of SDS-polyacrylamide gel electrophoresis (SDS-PAGE) analysis of TFF1 and TFF3 recombinant proteins (3 μg) reduced with 100 mM DTT, heat-denatured and electrophoresed. TFF3 possessed a molecular weight of approximately 9 kDa and TFF1 approximately 7 kDa.

FIG. 2 is a photograph depicting the results of Native-PAGE analysis of recombinant TFF1 and TFF3. 3 μg of TFF1 and TFF3 were loaded using native sample buffer. Lane A: TFF3; Lane B: TFF1; Lane C: molecular weight markers indicated. Under native conditions, TFF1 was observed predominantly as a single band (monomer) and a minor band (dimer) whereas TFF3 was observed as a dimer and at high molecular weight (tetramer).

FIG. 3 is a photograph depicting the results of Native-PAGE western blot analysis of recombinant human TFF1 and TFF3 proteins. TFF3 was observed to run as a multimeric form (˜35 kDa) and TFF1 was observed predominantly as a monomer and, to a lesser extent, a dimer. Lane A: recombinant TFF1 protein blotted with TFF3 antibodies; Lane B: recombinant TFF3 protein blotted with TFF3 antibodies; Lane C: recombinant TFF3 protein blotted with TFF1 antibodies; Lane D: recombinant TFF1 protein blotted with TFF1 antibodies; Lane E: Molecular weight markers are indicated. The polyclonal antibodies are highly specific against the antigens against which they were raised. The antibodies are highly sensitive to their antigens: the antibodies were used at 1:100,000 and still showed a strong signal and in ELISA; the antibodies titred at more than 1:5,000,000).

FIG. 4 is a graph illustrating that antibodies to either TFF1 or TFF3 produce apoptosis of mammary carcinoma cells in vitro. Percentage of cells undergoing apoptosis was determined by Hoescht 33258 visualization of apoptotic nuclei.

FIG. 5 is a graph photograph depicting the expression of TFF1 and TFF3 in a variety of human cancer cell lines as determined by reverse transcriptase PCR. Name of cells and tissue origin is indicated on the figure. B-Actin was used as a loading control.

FIG. 6 is a graph illustrating the effects of conformation specific TFF1 or TFF3 antibodies on the inhibition of survival of (A) mammary carcinoma cell; (B) prostate carcinoma cell and (C) gastric carcinoma cell and (D) T47D mammary carcinoma cell viability in vitro. For comparison, Tamoxifen (TAM) was also included in (A). (IgG=control IgG; pAb1=polyclonal antisera to TFF1; pAb3=polyclonal antisera to TFF3.)

FIG. 7 is a graph illustrating that the tumor volume formed by MCF-7 cells was reduced by polyclonal antibodies to TFF1 (A) or TFF3 (B) in a xenograft assay in immunocompromised mice. Arrow indicates start of administration of control IgG or antibodies directed to either TFF1 or TFF3.

FIG. 8 is a graph illustrating that mouse monoclonal antibodies to TFF1 reduced gastric carcinoma cell viability in vitro (A). The Figure also includes representative photomicrographs of the gastric carcinoma cells after 72 hours incubation with TFF1 monoclonal antibody 1C6 (B) compared to control IgG (C).

FIG. 9 is a graph illustrating that a mouse monoclonal antibody to TFF1 (IC6) reduced HT-29 colon carcinoma cell viability in vitro in comparison to control mouse IgG (mIgG).

FIG. 10 is a graph illustrating that a mouse monoclonal antibody to TFF1 reduced DLD-1 colon carcinoma cell viability in vitro.

FIG. 11 is a graph illustrating that a mouse monoclonal antibody to TFF1 reduced MCF-7 mammary carcinoma cell viability in vitro.

FIG. 12 is a graph illustrating that a mouse monoclonal antibody to TFF3 reduced DLD-1 colon carcinoma cell viability in vitro.

FIG. 13 is a graph illustrating that a mouse monoclonal antibody to TFF3 reduced HT-29 colon carcinoma cell viability in vitro.

FIG. 14 is a graph illustrating that a mouse monoclonal antibody to TFF3 reduced MCF-7 mammary carcinoma cell viability in vitro.

FIG. 15 is a graph illustrating that a mouse monoclonal antibody to TFF3 reduced T47D mammary carcinoma cell viability in vitro.

FIG. 16 is a graph illustrating detecting of TFF1 antigen using the 2D7 monoclonal antibody at varying concentrations. 10 and 100 ng of 2D7 mAb detected less than 0.1 pg of TFF1 antigen.

### DETAILED DESCRIPTION OF THE INVENTION

The trefoil factor family of proteins are characterized by a 40-amino acid trefoil motif that contains 3 conserved disulfide bonds. The 3 intrachain disulfide bonds form the trefoil motif (TFF domain). The trefoil motif is known in the art, e.g. Taupin and Podolsky, Nat Rev Mol Cell Bio. 4(9):721-32, 2003; Hoffmann et al., Histol Histopathol 16(1):319-34, 2001; and Thim, Cell Mol Life Sci 53(11-12):888-903, 1997.

In humans, three distinct members of the trefoil peptides have been identified. TFF1 or pS2 was first detected in a mammary cancer cell line as an estrogen-inducible gene. In human stomach, it is predominantly located in the foveolar cells of the gastric mucosa. TFF2 (formerly spasmolytic polypeptide or SP) was first purified from porcine pancreas and is expressed in mucous neck cells, deep pyloric glands, and Brunner's glands. TFF3 or intestinal trefoil factor (ITF) was the last to be identified and is predominantly expressed in the goblet cells of the small and large intestine. The trefoil peptides are involved in mucosal healing processes and are expressed at abnormal elevated levels in neoplastic diseases. A wide range of human carcinomas and gastrointestinal inflammatory malignancies, including peptic ulceration and colitis, Crohn's syndrome, pancreatitis, and biliary disease, aberrantly express trefoil peptides. Orthologues of these human proteins have been identified in other animals; for example, rats, mice and primates.

The trefoil family of peptides possess divergent function in the mammary gland with TFF1 functioning as a mitogen and TFF2 stimulating branching morphogenesis and cell survival. TFF3 is widely co-expressed with TFF1 in malignancies of the human mammary gland whereas TFF2 is not expressed in the mammary epithelial cells.

Reference herein to “TFF”, “TFF protein(s)”, or “TFF family of proteins” refers to the group of related proteins including TFF1, TFF2, and TFF3. TFF proteins share at least approximately 28 to 45% amino acid identity within the same species.

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM; preferably ≦100 nM and most preferably ≦10 nM.

Those skilled in the art will recognize that it is possible to determine, without undue experimentation, if an antibody has the same specificity as a TFF1 or TFF3 antibody described herein by ascertaining whether the former prevents the latter from binding to a CD3 antigen polypeptide. If the antibody being tested competes with an antibody of the invention, as shown by a decrease in binding by the TFF1 or TFF3 antibody of the invention, then the two antibodies bind to the same, or a closely related, epitope. Another way to determine whether an antibody has the specificity of an antibody of the invention is to pre-incubate the antibody of the invention with the TFF antigen with which it is normally reactive, i.e., TFF1 or TFF3, and then add the antibody being tested to determine if the antibody being tested is inhibited in its ability to bind the TFF antigen. If the antibody being tested is inhibited then, it is likely to have the same, or functionally equivalent, epitopic specificity as the antibody of the invention.

The data described herein were generated using the following materials and methods.

### Recombinant Human TFF1 and TFF3 Protein Production

The Glutathione S-transferase (GST) Gene Fusion System from Amersham Biosciences was used to produce recombinant TFF1 and TFF3 proteins in E. coli bacteria. The human TFF1 and TFF3 cDNA from mammary carcinoma (MCF-7) cells coding for mature proteins were amplified using reverse transcriptase-PCR(RT-PCR) with the following pairs of primers: 5′-ccg gaa ttc GAG GCC CAG ACA-3′ (forward, cloning linker in lower case) and 5′-ccg ctc gag CTA AAA TTC ACA-3′ (reverse) for TFF 1; and 5′-ccg gaa ttc GAG GAG TAC GTG GGC-3′ (forward) and 5′-ccg ctc gag TCA GAA GGT GCA-3′ (reverse) for TFF3. The RT-PCR products were cloned into pGEX 4T1 vector (Amersham Biosciences) by 5′ EcoRI and 3′ XhoI digestion to generate pGEX 4T1-TFF1 and pGEX 4T1-TFF3 plasmids. The plasmids were DNA sequence verified.

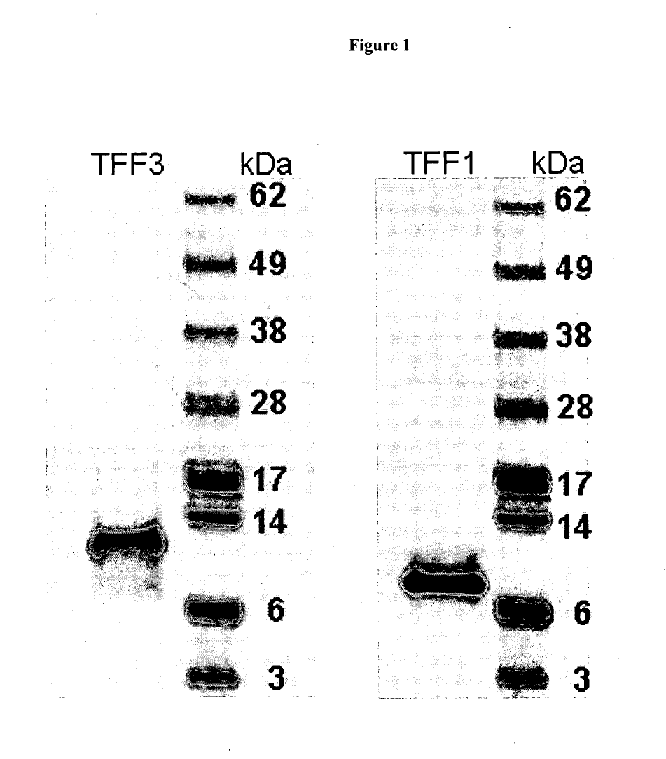

The pGEX 4T1-TFF1 or pGEX 4T1-TFF3 plasmids were used to transform BL21-Gold competent cells (Strategene). A single recombinant E. coli colony was inoculated into LB medium containing carbenicillin (50 μg/ml). The overnight culture was diluted 1:200 in LB medium/carbenicillin, pH 7.4, and cultured at 37° C. to optical density at 600 nm of ˜0.5. Protein expression was then induced by adding isopropyl-β-D-thiogalactopyranoside (IPTG) to a final concentration of 0.2 mM and the cultures were incubated for an additional 3-4 hours at 37° C. Cells were harvested and GST-fusion proteins were extracted from pellet under non-denaturing conditions and purified using glutathione sepharose 4B matrix (Amersham Biosciences). The GST fusion protein bound to the column was digested with thrombin protease to cleave TFF1/TFF3 proteins from GST and eluted from column with PBS to yield essentially pure product. The integrity of the GST fusion proteins and purified TFF1 and TFF3 proteins were analyzed by SDS-polyacrylamide gel electrophoresis (PAGE) on a 4-12% NuPAGE bis-tris gel (Invitrogen) and visualized by Coomassie Blue staining and quantified using Bradford's assay (FIG. 1).

The TFF1 and TFF3 recombinant proteins were successfully extracted from bacteria. Then proteins were then solubilized under native conditions and purified by affinity chromatography. The final native proteins were, therefore, suitable to be used as antigens for polyclonal antibody production. The protocol was up-scaled to produce milligrams of recombinant proteins required for production and affinity purification of the conformation specific polyclonal antibodies.

### Polyclonal Antibody Production

Five milligrams of purified recombinant native TFF1 and TFF3 proteins produced in E. coli were used to raise respective rabbit anti-TFF1 and TFF3 polyclonal antibodies. Two ZIKA female rabbits were immunized with 200 μg of each antigen by injecting intra muscularly into the animals. They were boosted every month with 100 μg of antigens. Rabbits were bled at the end of each month by catheterization of the marginal ear vein and the antibodies were affinity purified from the antiserum: anti-TFF1-06 and anti-TFF1-07, and anti-TFF3-08 and anti-TFF3-09. The sensitivity was measured using ELISA titration and the specificity was measured using native and reducing western blot analysis.

The conformation specific polyclonals antibodies were raised against native TFF1 and TFF3 antigens and affinity purified. All the batches of antibodies from the four rabbits were tested for their sensitivity/affinity by ELISA and specificity/antigen-selectivity by native western blot analysis.

### Deposit of Biological Materials

Under the terms of the Budapest Treaty on the International Recognition of the Deposit of Microorganisms for the Purpose of Patent Procedure, the hybridoma cell lines M661/7E5/1C6, M661/7E5/3F6, M661/7E5/2D7, M661/7E5/2C5, and M661/7E5/1D8, which produce antibodies referred to herein as 1C6, 3F6, 2D7, 2C5, and 1D8, were deposited on Sep. 27, 2007, with the American Type Culture Collection (ATCC) of 10801 University Boulevard, Manassas, Va. 20110-2209 USA under Federal Express Airbill Tracking Number 7991 9353 8311. Acknowledgment of receipt of the deposited cell lines on Sep. 27, 2007 was sent by the ATCC Patent Depository on Oct. 3, 2007.

### ELISA

100 ng of native TFF1 and TFF3 recombinant proteins were separately coated onto 96-well NUNC MaxiSorp microtitre plates in 100 t1 of 50 mM NaHCO₃/pH 8.5 and incubated overnight at RT. The wells were blocked with 5% non-fat dry milk powder in PBS/0.1% Tween-20 (PBST) for 1 hr. After blocking, 100 μl of primary antibody (polyclonal anti-TFF1 or anti-TFF3) was added to wells and incubated for 1 hour. The stock concentration of TFF1 and TFF3 polyclonal antibodies was 1 μg/μl, serially diluted using PBST: 1:500, 1:1000, 1:10000, 1:25000, 1:50000, 1:1000000, 1:2000000, 1:4000000 and 1:8000000 in triplicate. Afterwards the wells were washed 3 times with PBST and then incubated with HRP conjugated anti-rabbit secondary antibody (diluted 1:1000 in PBST) for 1 hr. The wells were washed 5 times with PBST and signal developed using substrate buffer. The reaction was stopped by adding 3N HCl. The plates were read at 405 nm.

The ELISA results showed that all four polyclonal antibodies have high affinity towards their respective native antigens and do not cross create with the other native trefoil factor antigen. The antibodies titrated out after to greater than out to 1:4000000 dilution. Native-PAGE and Native-Western Blot Analysis Recombinant proteins were electrophoresed under Native conditions using Novex® 4-20% tris-glycine pre-cast gels (Invitrogen) at 1 25V, until the Ponceau S tracking dye neared the bottom of the gel. Proteins were transferred to polyvinylidene difluoride (PVDF) membrane using tris/glycine transfer buffer at 25V, 2 hrs. Then the membrane was blocked using 5% non-fat dry milk in PBS-0.1% Tween-20 (PBST) for 2 hrs at RT. The membrane was incubated overnight at 4C with primary antibody (polyclonal TFF1 or TFF3) diluted accordingly in blocking buffer. After washing with PBST, the membrane was incubated with anti-rabbit IgG-HRP conjugated secondary antibody diluted with blocking buffer at RT for 1 h with gentle agitation. A further three 5 min washes with PBST buffer were carried out, then SuperSignal West Dura Extended Duration Substrate (Pierce) was added and membrane exposed to film.

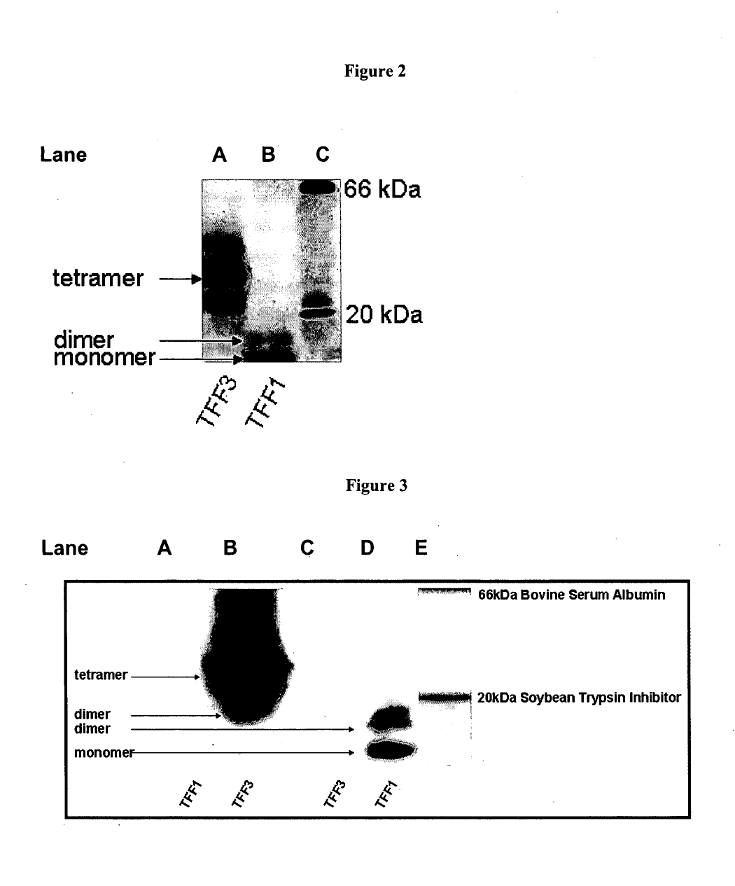

The native-PAGE demonstrated that recombinant native TFF 1 principally resolves as a single band (monomer) and a minor band (dimer) whereas recombinant TFF3 resolves as a single band (tetramer) and a minor band (dimer) (FIG. 2). The native western using polyclonal antibodies also confirmed that TFF1 predominantly forms a monomer and, minorly, a dimer whereas TFF3 forms a tetramer, approximately 35 kDa and a dimer (FIG. 3). The polyclonal antibodies demonstrated complete specificity towards their antigens and no cross reactivity with the other trefoil factor protein.

### In Silico Conformational Epitope (CE) Prediction

TFF1 and TFF3 proteins share high nucleotide sequence homology. Structures of human TFF1 and TFF3 have been resolved previously using NMR and the co-ordinates have been deposited at Protein Data Bank (PDB). NMR co-ordinates were fed into RASMOL, 3-D molecular visualization software. Homodimers formed by TFF1 and TFF3 show significant difference in the total structure and conformation. Epitopes are defined as the portions of the antigen molecules that interact with the antigen-binding site of an antibody. The Native western analysis presented herein demonstrates that the antibodies are specific and do not cross react with the other trefoil factor. Epitopes are of two types, namely, sequential epitope (SE) (when the antibody (Ab) binds to a contiguous stretch of amino acid residues that are linked by peptide bond) and conformational epitope (CE) (when Ab binds to non-contiguous residues, brought together by folding of polypeptide chain). Sequential epitopes are also referred to herein as linear epitopes. Antibodies that bind linear epitopes are Abs that bind to consecutive amino acids of an antigen sequence. Conformational epitopes are also referred to herein as native epitopes. Antibodies that bind conformational epitopes or native epitopes are Abs that bind TFF polypeptides and/or proteins in solution, e.g., physiologically compatible conditions. It is known from the analyses of the crystal structures of Ag-Ab complexes that, in order to be recognized by the antibodies, the residues must be accessible for interactions and thus be present on the surface of antigens. In addition, the conformation specific antibodies to TFF provided herein may bind to denatured TFF polypeptides or proteins or linear sequences derived from TFFs. A commercially available algorithm was used to predict SE and CE of TFF1 and TFF3. The results are shown below in Tables 1-6.

TABLE 1
Conformational epitopes predicted on the TFF1 homodimer.

| | | Res within |
|---|---|---|
| CE | | 6 Å of |
| No. | AD within 6 Å of Reference AD | Ref AD |
| 1 | A_1: EAQTETCTVApRErQN: 16 | A_43: W |
| | A_19: FPGvTPSQcANKG: 31 | |
| | A_34: FDDTVRG: 40 | |
| | A_46: YPnTIDVPPEEECEF: 60 | |
| | B_46: YPNTIDVPPEEECEF: 60 | |
| 2 | A_19: FPGvTPSQcANKG: 31 | A_43: W |
| | A_1: EAQTETCTVApRErQN: 16 | |
| | A_34: FDDTVRG :40 | |
| | A_46: YPnTIDVPPEEECEF: 60 | |
| 3 | A_34: FDDTVRG: 40 | A_43: W |
| | A_1: EAQTETCTVApRErQN: 16 | |
| | A_19: FPGvTPSQcANKG: 31 | |
| 4 | A_46: YPnTIDVPPEEECEF: 60 | |
| | A_1: EAQTETCTVApRErQN: 16 | |
| | A_19: FPGvTPSQcANKG: 31 | |
| | A_46: YPNTIDVPPEEECEF: 60 | |
| 5 | B_1: EAQTETcTvAPRErQNcGFPGvTPSQcANKG: | |
| | 31 | |
| | B_34: FdDTVRG: 40 | |
| | B_46: YPNTIDVPPEEECEF: 60 | |
| 6 | B_46: FdDTVRG: 40 | |
| | B_1 : EAQTETcTVAPRErQNcGFPGvTPSQcANKG: | |
| | 31 | |
| 7 | B_46: YPNTIDVPPEEECEF: 60 | |
| | A_1: EAQTETCTVApRErQN: 16 | |
| | A_46: YPnTIDVPPEEECEF: 60 | |
| | B_1: EAQTETcTvAPRErQNcGFPGvTPSQcANKG: | |
| | 31 | |

AD—antigenic determinants; CE—conformational epitope; A and B are two TFF1 proteins forming the homodimer.

Table 1 shows the seven different conformational epitopes present in TFF1 homodimer. Any AD found within 6 Å distance to the reference AD forms a part of a conformational epitope. There were no sequential epitopes (SE) predicted. Upper case shows the amino-acids available at the surface and lower case shows the amino acids embedded within the tertiary structure and having less than 25% accessibility to the antibody.

TABLE 2
Antigenic determinants predicted on TFF1 homodimer.
PREDICTED AD

| AD No. | Antigenic Determinant |
|---|---|
| 1_A | A_1: EAQTETCTVApRErQN: 16 |
| 2_A | A_19: FPGvTPSQcANKG: 31 |
| 3_A | A_34: FDDTVRG: 40 |
| 4_A | A_46: YPnTIDVPPEEECEF: 60 |
| 5_B | B_1: EAQTETcTvAPRErQNcGFPGvTPSQcANKG: 31 |
| 6_B | B_34: FdDTVRG: 40 |
| 7_B | B_46: YPNTIDVPPEEECEF: 60 |

AD—antigenic determinants; A and B are two TFF1 proteins forming the homodimer.

Table 2 shows the seven different antigen determinants (AD) found in the native TFF 1 homodimer. The AD is from 7 to 31 amino acids in length spanning more than 80% of the primary protein sequence is involved in forming different conformational epitopes.

TABLE 3
Conformational epitopes predicted on TFF1 monomer.

| | | Res within |
|---|---|---|
| CE | Predicted CE formed by | 6 Å of |
| No. | AD within 6 Å of Reference AD | Ref AD |
| 1 | A_1: EAQTETCTVApRErQN: 16 | A_43: W |
| | A_19: FPGvTPSQcANKG: 31 | |
| | A_34: FDDTVRG: 40 | |
| | A_46: YPnTIDVPPEEECEF: 60 | |
| 2 | A_34: FDDTVRG: 40 | A_43: W |
| | A_1: EAQTETCTVApRErQN: 16 | |
| | A_19: FPGvTPSQcANKG: 31 | |
| 3 | A_46: YPnTIDVPPEEECEF: 60 | |
| | A_1: EAQTETCTVApRErQN: 16 | |
| | A_19: FPGvTPSQcANKG: 31 | |

AD—antigenic determinants; CE—conformational epitope.

Table 3 shows the three different conformational epitopes present in TFF1 monomer. Any AD found within 6 Å distance to the reference AD forms a part of a conformational epitope. There were no sequence epitopes found. Upper case shows the amino-acids available at the surface and lower case shows the amino acids embedded within the tertiary structure and having less than 25% accessibility to the antibody.

TABLE 4
Antigenic determinants predicted on TFF1 monomer.
PREDICTED AD

| AD No. | Antigenic Determinant |
|---|---|
| 1_A | A_1: EAQTETCTVApRErQN-16 |
| 2_A | A_19: FPGvTPSQcANKG: 31 |
| 3_A | A_34: FDDTVRG-40 |
| 4_A | A_46: YPnTIDVPPEEECEF-60 |

AD—antigenic determinants.

Table 4 shows the four different antigen determinants (AD) found in the TFF1 homodimer. The AD is from 7 to 16 amino acids in length spanning more than 80% of the primary protein sequence is involved in forming different conformational epitopes.

TABLE 5
Conformational epitopes predicted on TFF3 homodimer.

| | | Res within |
|---|---|---|
| CE | | 6 Å of |
| No. | AD within 6 Å of Reference AD | Ref AD |
| 1 | A_1: EEYVTLsAN: 9 | A_52: L |
| | A_12: AvPaKDrVDcGyPHvTPKEcNN: 33 | A_59: F |
| | B_1: EEYVGLsAN: 9 | |
| 2 | A_12: AvPaKDrVDcGyPHvTPKEcNN: 33 | A_43: W |
| | A_1: EEYVGLsAN: 9 | |
| | A_40: SRIPG: 44 | |
| | B_12: AvPaKDrVDcGyPHVTPKEcNN: 33 | |
| 3 | A_40: SRIPG: 40 | A_43: W |
| | A_12: AvPaKDrVDcGyPHVTPKEcNN: 33 | |
| 4 | B_1: EEYVGLsAN: 9 | |
| | B_1: EEYVGLsAN: 9 | |
| | B_12: AvPaKDrVDcGyPHVTPKEcNN: 33 | |
| 5 | B_12: AvPaKDrVDcGyPHvTPKEcNN: 33 | |
| | A_12: AvPaKDrVDcGyPHvTPKEcNN: 33 | |
| | B_38: FdSRIPG: 44 | |
| | B_1: EEYVGLsAN: 9 | |
| 6 | B_38: FdSRIPG: 44 | |
| | B_12: AvPaKDrVDcGyPHvTPKEcNN: 33 | |

AD—antigenic determinants; CE—conformational epitope; A and B are two TFF3 proteins forming the homodimer.

Table 5 shows the six different conformational epitopes present in TFF3 homodimer. Any AD found within 6 Å distance to the reference AD forms a part of a conformational epitope. Similar to TFF1 homodimer there were no sequential epitope (SE) found. None of the CE was found overlapping with TFF1 homodimer. Upper case shows the amino-acids available at the surface and lower case shows the amino acids embedded within the tertiary structure and having less than 25% accessibility to the antibody.

TABLE 6
Antigenic determinants predicted on TFF3 homodimer.
PREDICTED AD

| AD No. | Antigenic Determinant |
|---|---|
| 1_A | A_1: EEYVTLsAN: 9 |
| 2_A | A_12: AvPaKDrVDcGyPHvTPKEcNN: 33 |
| 3_A | A_40: SRIPG: 44 |
| 4_A | B_1: EEYVGLsAN: 9 |
| 5_A | B_12: AvPaKDrVDcGyPHVTPKEcNN: 33 |
| 6_A | B_38: FdSRIPG: 44 |

AD—antigenic determinants; A and B are the two TFF3 molecules forming homodimer.

Table 6 shows the six different antigen determinants (AD) found in the native TFF1 homodimer. The AD is from 5 to 21 amino acids in length spanning 50% of the primary sequence of the protein involved in forming different conformational epitopes.

The monomeric and dimeric forms of the native TFF1 and dimeric form of TFF3 NMR resolved 3D structures were used to predict their epitopes. Both native TFF1 and TFF3 have multiple conformational epitopes (CE) spanning the exposed native surface structure and do not posses any sequential epitopes (SE). The two antigens exhibit different CE's concordant with the antigen specificity of the antibodies observed in native western. TFF1 monomer and dimer share two epitopes in common. TFF1 and TFF3 do not share any antigenic determinants (AD) in common.

### Monoclonal Antibody Production

Mice were immunized by injection of native TFF1 or TFF3 proteins. Monoclonal antibodies were produced according to standard methodologies. Hybridoma clones were expanded in culture to produce large quantities of a single type of antibody developed against a conformational epitope(s). These antibodies, called monoclonal antibodies, were examined for their specificity and sensitivity using ELISA.

MTT (3-(4,5-dimethylthiazol-2-yl)-2,5-diphenyl tetrazolium bromide) Assay

A rapid colorimetric assay, based on the tetrazolium salt MTT (3-(4,5-dimethylthiazol-2-yl)-2,5-diphenyl tetrazolium bromide), that measures only living cells and can be read on a scanning multiwell spectrophotometer (ELISA reader). MTT is converted to purple formazan only when mitochondrial reductase enzymes are active, and thus conversion is directly related to the number of viable cells. The production of formazan in cells treated with an agent is measured relative to the production in control cells, and a dose-response curve can be generated.

Various cancer cell lines were purchased from the ATCC and seeded at a density of 2500 cells/100 μl media in each well of a 96-well plate. Cells were treated with different concentration of either polyclonal rabbit or mouse monoclonal TFF1 and TFF3 antibodies (final volume 100 μl). The same procedure was performed with the same concentration of rabbit IgG (for polyclonal TFF1 and TFF3 antibodies) or mouse IgG (for mouse monoclonal TFF1 and TFF3 polyclonal antibodies) and utilized as control. Microplates were incubated at 37° C. in a 5% CO₂ atmosphere for 72 h. Subsequently, 20 μl of MTT (5 mg/ml) solution was added to each well of 96-well plates containing cells treated with different concentrations of antibodies. The reaction was stopped after 4 h incubation by addition of 140 μl of 0.04 mol/L HCl in isopropanol and the absorbance of each well was measured by an ELISA reader using a test wavelength of 490 nm. Each concentration treatment was done in triplicate wells.

### Apoptosis Assay

Apoptotic cell death in the presence or absence of antibodies to TFF1 or TFF3 was measured by fluorescent microscopic analysis of cell DNA staining patterns with karyophilic Hoechst 33258 (Del, B. G., Z. Darzynkiewicz, C. Degraef, R. Mosselmans, D. Fokan, and P. Galand. 1999.

Diagnosis of Cancers and/or Proliferation Disorders

The conformation specific antibodies to TFF described herein are also useful in a variety of diagnostic applications. The invention features a method for diagnosing cancer or a cell proliferation and/or survival disorder in a mammal by contacting a tissue or bodily fluid from the mammal with a conformational specific antibody to TFF under conditions sufficient to form an antigen-antibody complex and detecting the antigen-antibody complex. Cancers or tumors detected using the conformation specific antibodies to TFF described herein include an epithelial tumor such as, e.g., lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor. Proliferative disorders detected using the conformation specific antibodies to TFF described herein include, e.g., keratinocyte hyperproliferation, inflammatory cell infiltration, cytokine alteration, endometriosis, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

Methods for diagnosis include detecting a tumor cell in vivo or ex vivo in bodily fluids or in tissue. For example, a biopsied tissue is contacted with an antibody and antibody binding measured. In addition to biopsied tissue samples, whole blood, serum, plasma, stool, urine, cerebrospinal fluid, bronchoalveolar lavage, sputum, exhaled breath condensate, semen, saliva, joint fluid or ulcer secrete is tested. Whole body diagnostic imaging may be carried out to detect microtumors undetectable using conventional diagnostic methods. Accordingly, a method for diagnosing a tumor in a mammal is carried out by contacting a tissue, e.g., a lymph node, of a mammal with a detectably-labeled antibody which binds to a TFF conformational epitope. An increase in the level of antibody binding at a tissue site compared to the level of binding to a normal nonneoplastic tissue indicates the presence of a neoplasm at the tissue site. For detection purposes, the antibody is labeled with a detectable marker, e.g., non-radioactive tag, a radioactive compound, or a colorimetric agent. For example, the antibody or antibody fragment is tagged with ¹²⁵I, ⁹⁹Tc, Gd⁺⁺⁺, or Fe⁺⁺. Green fluorescent protein is used as a colorimetric tag.

A method for diagnosis or prognosis is carried out by contacting a bodily fluid or tissue sample from the mammal with an antibody under conditions sufficient to form an antigen-antibody complex and detecting the antigen-antibody complex; quantitating the amount of complex to determine the level of TFF, and comparing the level with a normal control level of TFF. For prognostic purposes, an increasing level of TFF over time indicates a progressive worsening of the disease, and therefore, an adverse prognosis. Patient derived tissue samples, e.g., biopsies of solid tumors, as well as bodily fluids such as a CNS-derived bodily fluid, blood, serum, urine, saliva, sputum, lung effusion, and ascites fluid, are contacted with a conformation specific antibodies to TFF.

Detecting an increase in TFF or TFF gene products in a patient-derived tissue sample (e.g., solid tissue or bodily fluid) is carried out using standard methods, e.g., by Western blot assays or a quantitative assay such as ELISA. For example, a standard competitive ELISA format using a conformation specific antibody to TFF is used to quantify patient TFF levels. Alternatively, a sandwich ELISA using a first antibody as the capture antibody and a second conformation specific antibody as a detection antibody is used.

Methods of detecting TFF include contacting a component of a bodily fluid with a conformation specific antibody bound to solid matrix, e.g., microtiter plate, bead, dipstick. For example, the solid matrix is dipped into a patient-derived sample of a bodily fluid, washed, and the solid matrix is contacted with a reagent to detect the presence of immune complexes present on the solid matrix.

Proteins in a test sample are immobilized on (e.g., bound to) a solid matrix. Methods and means for covalently or noncovalently binding proteins to solid matrices are known in the art. The nature of the solid surface may vary depending upon the assay format. For assays carried out in microtiter wells, the solid surface is the wall of the microtiter well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates, polyvinylidine fluoride (known as IMMULON™), diazotized paper, nylon membranes, activated beads, and Protein A beads. The solid support containing the antibody is typically washed after contacting it with the test sample, and prior to detection of bound immune complexes. Incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

A TFF-detection reagent, e.g., a conformation specific antibody, is packaged in the form of a kit, which contains one or more conformation specific antibodies, control formulations (positive and/or negative), and/or a detectable label. The assay may be in the form of a standard two-antibody sandwich assay format known in the art.

### EXAMPLES

TFF1 and TFF3 are overexpressed in cancer cells of various organs and induce invasion, survival and proliferation of neoplastic cells. The studies described herein were designed to evaluate the neutralization of secreted TFF1 and TFF3 using TFF1 and/or TFF3 specific antibodies as cytotoxic agents on cancer cells.

### Example 1

### Inhibition of Cell Survival in Human Colon Cancer Cell Lines by Conformation Specific Polyclonal TFF 1 and TFF3 Antibodies

The conformation specific TFF1 and TFF3 antibodies inhibited survival of HT-29 colon carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of COLO-320DM colon carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of DLD-19 colon carcinoma cells. Cell survival in response to treatment with these antibodies was determined by MTT assay, as described above. Each point represents mean ±Standard Error (SE) of triplicate determinations.

### Example 2

### Inhibition of Cell Survival in Human Ovarian Cancer Cell Lines by Conformation Specific Polyclonal TFF1 and TFF3 Antibodies

The conformation specific TFF1 and TFF3 antibodies inhibited survival of OVCAR-4 ovarian carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of OVCAR-5 ovarian carcinoma cells. Cell survival in response to treatment with these antibodies was determined by MTT assay, as described above. Each point represents mean ±Standard Error (SE) of triplicate determinations.

The TFF1 and TFF3 antibodies inhibited survival of OVCAR-8 ovarian carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of SKOV-3 ovarian carcinoma cells. Cell survival in response to treatment with these antibodies was determined by MTT assay, as described above. Each point represents mean ±Standard Error (SE) of triplicate determinations.

### Example 3

### Inhibition of Cell Survival Tumor Formation In Vitro and Anchorage-Independent Growth of Human Mammary Carcinoma Cell Lines by Conformation Specific Polyclonal TFF1 and TFF3 Antibodies

The conformation specific TFF1 and TFF3 antibodies inhibited survival of MCF-7 mammary carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited anchorage-independent growth of MCF-7 mammary carcinoma cells. Anchorage-independent growth of cells in response to treatment with these antibodies was determined by soft agar assay, as described above. Each point represents mean ±Standard Error (SE) of triplicate determinations.

The conformation specific TFF1 and TFF3 antibodies inhibited formation of tumor by MCF-7 mammary carcinoma cells in Matrigel. One week post-treatment, in vitro tumor regression was studied using phase contrast microscopy by morphological examination of colony sizes.

The conformation specific TFF1 and TFF3 antibodies inhibited survival of T47D mammary carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited anchorage-independent growth of T47D mammary carcinoma cells. Anchorage-independent growth of cells in response to treatment with these antibodies was determined by soft agar assay, as described above. Each point represents mean ±Standard Error (SE) of triplicate determinations.

The conformation specific TFF1 and TFF3 antibodies inhibited formation of tumor by T47D mammary carcinoma cells in Matrigel. One week post-treatment, in vitro tumor regression was studied using phase contrast microscopy by morphological examination of colony sizes.

The conformation specific TFF1 and TFF3 antibodies inhibited survival of MDA-MB-231 mammary carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of BT-549 mammary carcinoma cells. Cell survival in response to treatment with these antibodies was determined by MTT assay, as described above. Each point represents mean ±Standard Error (SE) of triplicate determinations.

### Example 4

### Inhibition of Cell Survival in Human Stomach Cancer Cell Line by Conformation Specific Polyclonal TFF1 and TFF3 Antibodies

The TFF1 and TFF3 antibodies inhibited survival of AGS gastric carcinoma cells. Cell survival in response to treatment with these antibodies was determined by MTT assay, as described above. Each point represents mean ±Standard Error (SE) of triplicate determinations.

### Example 5

### Inhibition of Cell Survival in Human Lung Cancer Cell Lines by Conformation Specific TFF1 and TFF3 Antibodies

The conformation specific TFF1 and TFF3 antibodies inhibited survival of A549 lung carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of NCI-H1299 lung carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of NCI-H2009 lung carcinoma cells. Cell survival in response to treatment with these antibodies was determined by MTT assay, as described above. Each point represents mean ±Standard Error (SE) of triplicate determinations.

### Example 6

### Inhibition of Cell Survival in Human Pancreatic Cancer Cell Lines by Conformation Specific Polyclonal TFF1 and TFF3 Antibodies

The conformation specific TFF1 and TFF3 antibodies inhibited survival of BX-PC3 pancreas carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of HPAC pancreas carcinoma cells. Cell survival in response to treatment with these antibodies was determined by MTT assay, as described above. Each point represents mean ±Standard Error (SE) of triplicate determinations.

### Example 7

### Inhibition of Cell Survival in Human Prostate Cancer Cell Lines by Conformation Specific Polyclonal TFF1 and TFF3 Antibodies

The conformation specific TFF1 and TFF3 antibodies inhibited survival of DU 145 prostate carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited survival of 22R VI prostate carcinoma cells. Cell survival in response to treatment with these antibodies was determined by MTT assay, as described above. Each point represents mean ±Standard Error (SE) of triplicate determinations.

### Example 8

### Inhibition of Cell Survival in Human Liver Cancer Cell Lines by Conformation Specific Polyclonal TFF1 and TFF3 Antibodies

The conformation specific TFF1 and TFF3 antibodies inhibited survival of SK-HEP-1 hepatic carcinoma cells. Cell survival in response to treatment with these antibodies was determined by MTT assay, as described above. Each point represents mean ±Standard Error (SE) of triplicate determinations.

### Example 9

### Inhibition of Cell Survival Tumor Formation In Vitro and Anchorage-Independent Growth of Human Endometrial Carcinoma Cell Lines by Conformation Specific Polyclonal TFF1 and TFF3 Antibodies

The conformation specific TFF1 and TFF3 antibodies inhibited anchorage-independent growth of AN3 CA endometrial carcinoma cells. Anchorage-independent growth of cells in response to treatment with these antibodies was determined by soft agar assay, as described above. Each point represents mean ±Standard Error (SE) of triplicate determinations.

The conformation specific TFF1 and TFF3 antibodies inhibited formation of tumor by AN3 CA endometrial carcinoma cells in Matrigel. One week post-treatment, in vitro tumor regression was studied using phase contrast microscopy by morphological examination of colony sizes.

The conformation specific TFF1 and TFF3 antibodies inhibited survival of RL95-2 endometrial carcinoma cells. Cell survival in response to treatment with these antibodies was determined by MTT assay, as described above. Each point represents mean ±Standard Error (SE) of triplicate determinations.

The conformation specific TFF1 and TFF3 antibodies inhibited formation of tumor by RL95-2 endometrial carcinoma cells in Matrigel. One week post-treatment, in vitro tumor regression was studied using phase contrast microscopy by morphological examination of colony sizes.

### Example 10

### Effect of Conformation Specific Polyclonal TFF1 Antibody on Tumor Volume and Reduction Thereof

Xenograft Analyses: MCF-7 (5×10⁶) cells were suspended in Matrigel and injected into the first mammary (axillary) fat pad of 3- to 4-week-old athymic (nu/nu) female nude mice treated either with TFF1 antibody (n=5, dark circle) or control mouse IgG (n=5, open circle). TFF1 antibody (200 μg) was delivered by i.p. injection every day for 2 weeks. The mice simultaneously received a 60-day release pellet containing 0.72 mg of 17β-estradiol (Innovative Research of America, Southfield, Mich.).

The tumor volume was measured on days 16, 19, 22, 15 and mice sacrificed at the end of the experiment at day 30. At necropsy, primary tumors and all organs were evaluated macroscopically for the presence of tumors. Tissue samples of the primary tumor and organs were fixed in 4% paraformaldehyde and stained with H&E to assess morphology.

Significant reduction in the tumor volume in the TF-1 antibody treated group was observed in comparison to the IgG-treated group (p<0.001).

### Example 11

### Antibodies to TFF1 and TFF3 produce apoptosis of MCF-7 mammary carcinoma cells

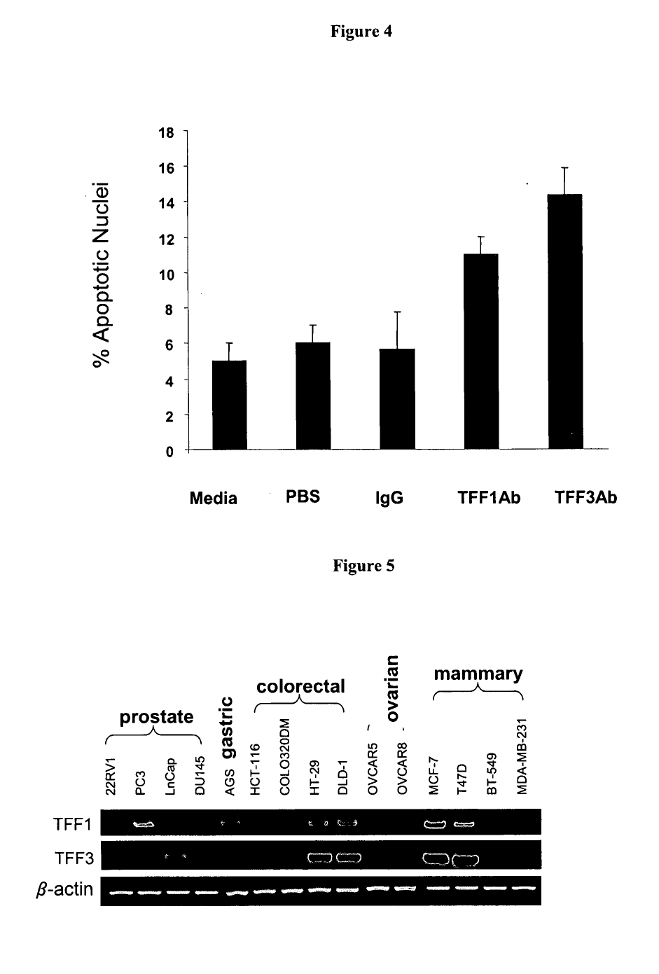

The conformation specific TFF1 (α-TFF-1 pAb) and TFF3 (α-TFF-3 pAb) polyclonal antibodies increased the apoptotic rate of MCF-7 mammary carcinoma cells (FIG. 4). Apoptosis in response to treatment with these antibodies in serum containing media was determined by Hoescht 33258 after 72 hour treatment of MCF-7 cells with antibodies. Rabbit IgG was used as control. Each point represents mean ±Standard Error (SE) of triplicate determinations.

### Example 12

### Expression of TFF1 and TFF3 in a Variety of Human Cancer Cell Lines

The expression of TFF-1 and TFF-3 proteins in a number of human cell lines was determined using reverse transcriptase PCR. The expression was examined in prostate (22RV1, PC3, LnCap and DU145), gastric (AGS), colorectal (HCT-116, COLO320DM, HT-29 and DLD-1), ovarian (OVCAR5 and OVCAR8) and mammary (MCF-7, T47D, BT-549 and MDA-MB-231) human cancer lines. Beta-Actin was used as a loading control (FIG. 5).

### Example 13

### Inhibition of Cell Survival in Human Cancer Cell Lines by Conformation Specific Polyclonal TFF1 and TFF3 Antibodies

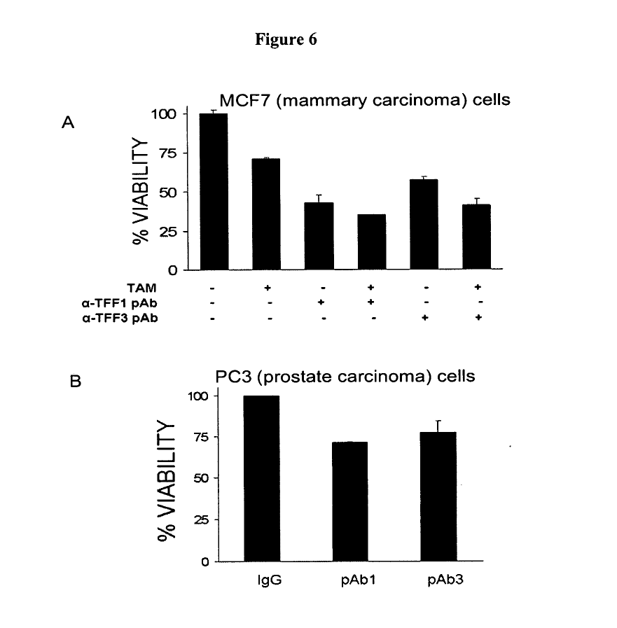

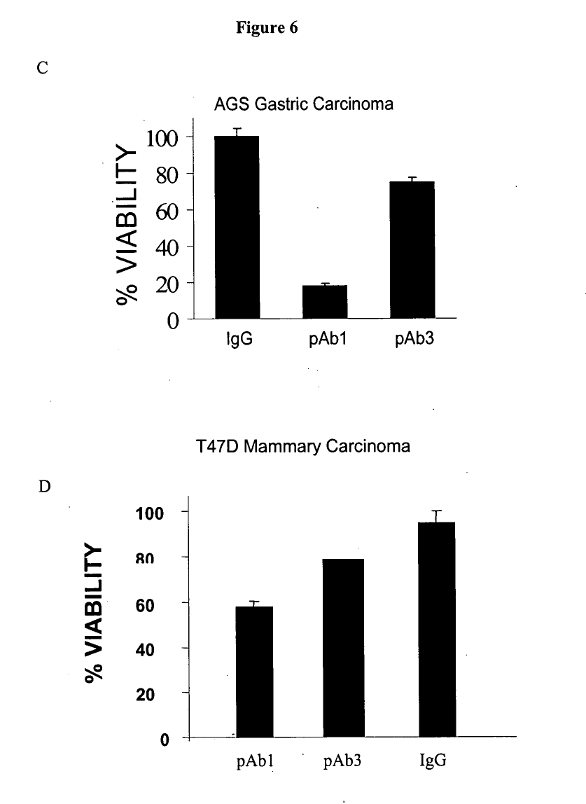

The conformation specific TFF1 (α-TFF-1 pAb) and TFF3 (α-TFF-3 pAb) polyclonal antibodies inhibited survival of MCF-7 mammary carcinoma cells (FIG. 6(A)), PC3 prostate carcinoma cells (FIG. 6(B)), AGS gastric carcinoma cells (FIG. 6(C)) and T47D mammary carcinoma cells (FIG. 6(D)). Cell survival in response to treatment with these antibodies was determined by MTT assay, as described above. Rabbit IgG was used as control. Each point represents mean ±Standard Error (SE) of triplicate determinations.

### Example 14

### Effect of Conformation Specific Polyclonal TFF1 Antibody on Tumor Volume and Reduction Thereof

Xenograft Analyses: MCF-7 (5×10⁶) cells were suspended in Matrigel and injected into the first mammary (axillary) fat pad of 3- to 4-week-old athymic (nu/nu) female nude mice treated either with TFF1 antibody (n=5, dark circle) or control mouse IgG (n=5, open circle). TFF1 antibody (200 μg) was delivered by i.p. injection every day for 2 weeks. The mice simultaneously received a 60-day release pellet containing 0.72 mg of 17β-estradiol (Innovative Research of America, Southfield, Mich.).

The tumor volume was measured on days 16, 19, 22, 15 and mice sacrificed at the end of the experiment at day 30.

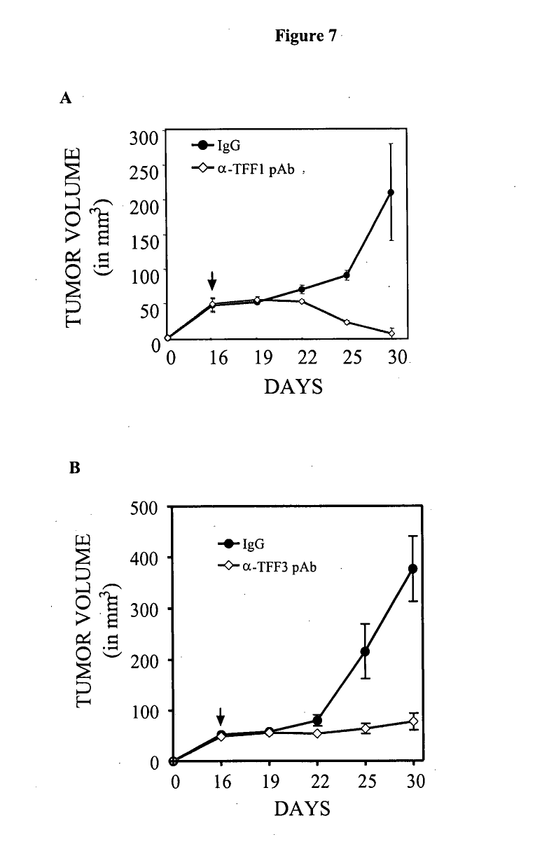

Polyclonal antibodies to TFF1 (FIG. 7(A)) or TFF-3 (7(B)) reduced mammary carcinoma (MCF-7) xenograft growth in immunocompromized mice. Arrow indicates start of administration of control IgG or antibodies directed to either TFF-1 or TFF-3.

### Example 15

### Inhibition of Cell Survival in Human Cancer Cell Lines by Conformation Specific TFF1 Monoclonal Antibodies

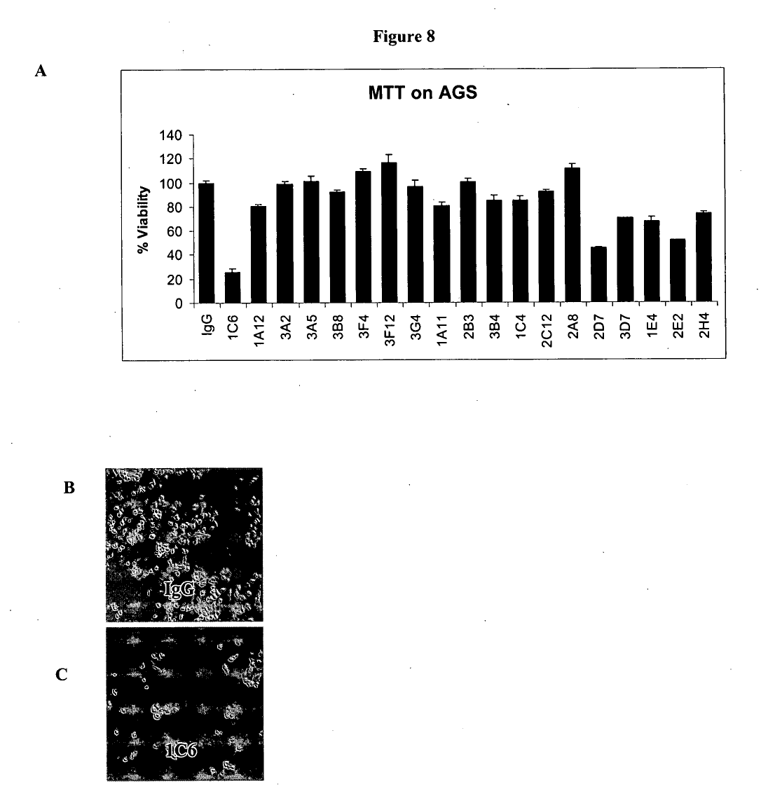

The tested mouse monoclonal antibodies to TFF1 (1C6, 1A12, 3A2, 3A5, 3B8, 3F4, 3F12, 3G4, 1A11, 2B3, 3B4, 1C4, 2C12, 2A8, 2D7, 1E4, 2E2, 2H4 (FIG. 8(A)) and 1C6, 3F6, 2C5, 3F11, 3F3, (FIG. 8(B)) and reduced gastric carcinoma cell viability in vitro. The most dramatic inhibitory effect was observed following the incubation of the cells with TFF1 1C6 monoclonal antibody. Photomicrographs (C) and (D) illustrate the efficacy of TFF1 monoclonal antibody 1C6 (D) following 72-hour incubation in comparison with the control IgG group (C). Cell survival in response to treatment with these antibodies was determined by MTT assay, as described above. Each point represents mean ±Standard Error (SE) of triplicate determinations.

### Example 16

### Inhibition of Cell Survival in Human Cancer Cell Lines by Conformation Specific TFF1 Mouse Monoclonal Antibody 1C6

Cell survival in response to treatment with the antibody was determined by MTT assay, as described above. Each point represents mean ±Standard Error (SE) of triplicate determinations.

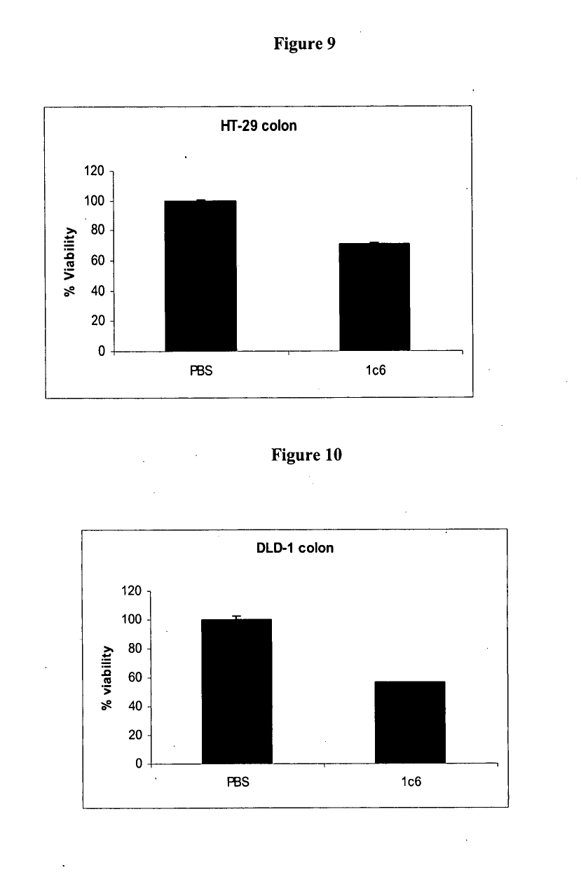

The 1C6 mouse monoclonal TFF1 antibody significantly reduced HT-29 colon carcinoma cell viability in vitro (FIG. 9).

The 1C6 mouse monoclonal TFF1 antibody significantly reduced DLD-1 colon carcinoma cell viability in vitro (FIG. 10).

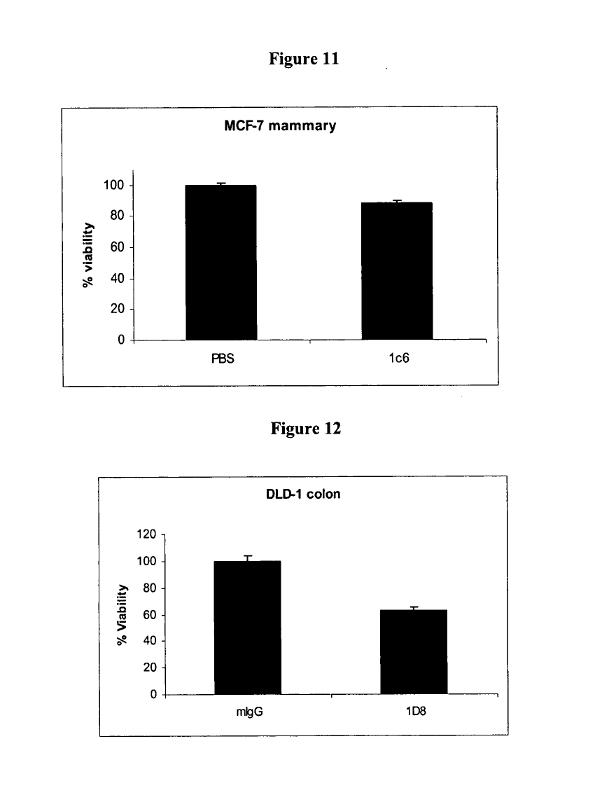

The 1C6 mouse monoclonal TFF1 antibody significantly reduced MCF-7 mammary carcinoma cell viability in vitro (FIG. 11).

### Example 17

### Inhibition of Cell Survival in Human Cancer Cell Lines by Conformation Specific TFF3 Mouse Monoclonal Antibody 1D8

Cell survival in response to treatment with the antibody was determined by MTT assay, as described above. Each point represents mean ±Standard Error (SE) of triplicate determinations.

The 1D8 mouse monoclonal TFF3 antibody significantly reduced DLD-1 colon carcinoma cell viability in vitro (FIG. 12).

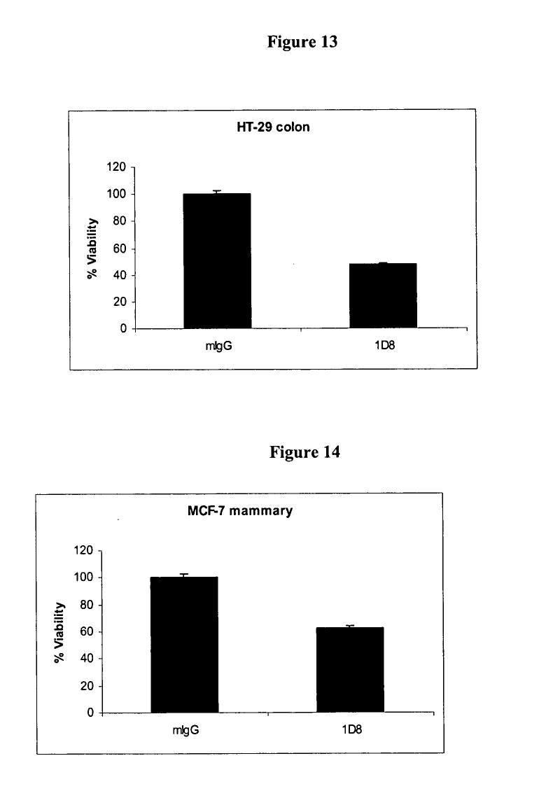

The 1D8 mouse monoclonal TFF3 antibody significantly reduced HT-29 colon carcinoma cell viability in vitro (FIG. 13).

The 1D8 mouse monoclonal TFF3 antibody significantly reduced MCF-7 mammary carcinoma cell viability in vitro (FIG. 14).

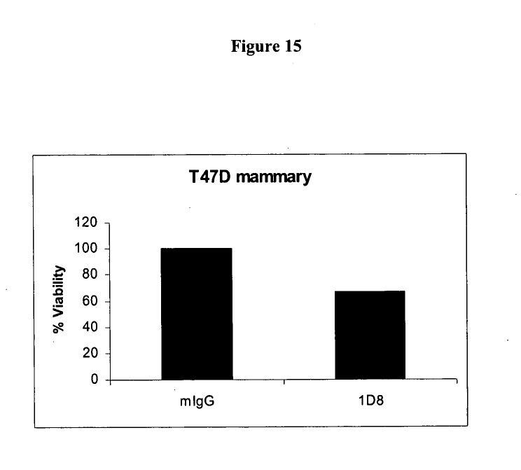

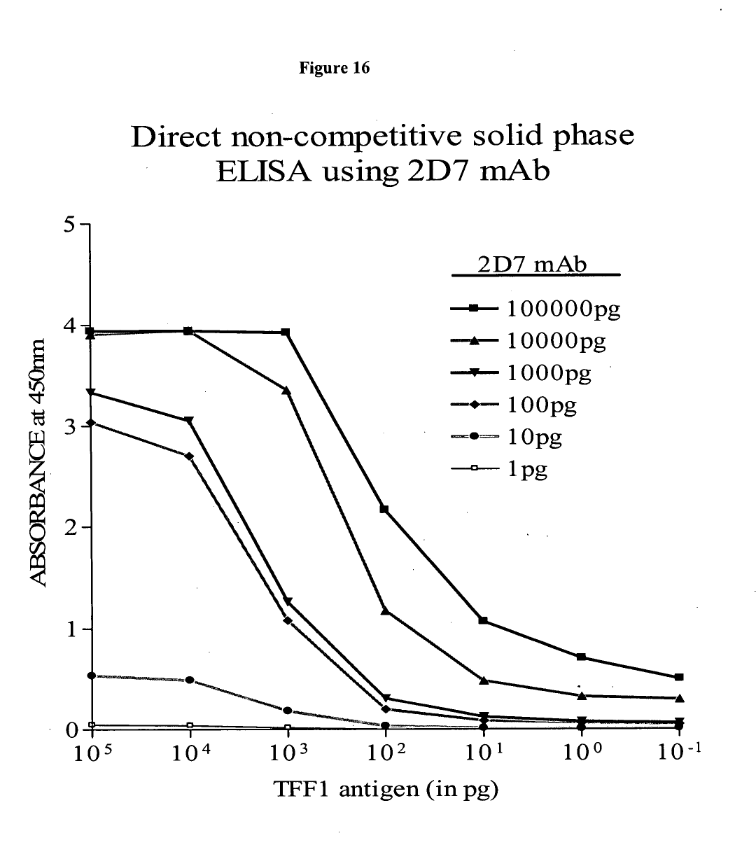

The 1D8 mouse monoclonal TFF3 antibody significantly reduced T47D mammary carcinoma cell viability in vitro (FIG. 15).

### Administration of Compositions for Cancer Therapy

The conformation specific TFF-specific antibodies described herein are used to inhibit the growth of a tumor cell, or kill the tumor cell. In addition to cancer therapy, the methods are useful to confer clinical benefit to those suffering from or at risk of developing a precancerous condition or lesion or a non-cancerous hyperproliferative disorder.

The agent (e.g., peptides or nucleic acids of the invention) of use in inhibiting a TFF may be used on their own, or in the form of compositions in combination with one or more pharmaceutically acceptable diluents, carriers and/or excipients.

As-used herein, the phrase “pharmaceutically acceptable diluents, carriers and/or excipients” is intended to include substances that are useful in preparing a pharmaceutical composition, may be co-administered with an agent in accordance with the invention while allowing same to perform its intended function, and are generally safe, non-toxic and neither biologically nor otherwise undesirable. Examples of pharmaceutically acceptable diluents, carriers and/or excipients include solutions, solvents, dispersion media, delay agents, emulsions and the like. Diluents, carriers and/or excipients may contain minor amounts of additives such as substances that enhance isotonicity and chemical stability.

A variety of pharmaceutically acceptable diluents, carriers and/or excipients known in the art may be employed in compositions of the invention. As will be appreciated, the choice of such diluents, carriers and/or excipients will be dictated to some extent by the nature of the agent to be used, the intended dosage form of the composition, and the mode of administration thereof. By way of example, in the case of administration of nucleic acids such as vectors adapted to express antisense or iRNA, suitable carriers include isotonic solutions, water, aqueous saline solution, aqueous dextrose solution, and the like.

In addition to standard diluents, carriers and/or excipients, a pharmaceutical composition of the invention may be formulated with additional constituents, or in such a manner, so as to enhance the activity of the agent or help protect the integrity of the agent. For example, the composition may further comprise adjuvants or constituents which provide protection against degradation, or decrease antigenicity of an agent, upon administration to a subject. Alternatively, the agent may be modified so as to allow for targeting to specific cells, tissues or tumors.

Additionally, the antibodies are formulated with other ingredients which may be of benefit to a subject in particular instances. For example, optionally, one or more anti-neoplastic agents are co-administered or incorporated into the formulation. Examples of such agents include: alkylating agents (e.g., chlorambucil (Leukeran™), cyclophosphamide (Endoxan™, Cycloblastin™, Neosar™, Cyclophosphamide™), ifosfamide (Holoxan™, Ifex™, Mesnex™), thiotepa (Thioplex™, Thiotepa™)); antimetabolites/S-phase inhibitors (e.g., methotrexate sodium (Folex™, Abitrexate™, Edertrexate™), 5-fluorouracil (Efudix™, Efudex™), hydroxyurea (Droxia™, Hydroxyurea, Hydrea™), amsacrine, gemcitabine (Gemzar™), dacarbazine, thioguanine (Lanvis™)); antimetabolites/mitotic poisons (e.g., etoposide (Etopophos™, Etoposide, Toposar™), vinblastine (Velbe™, Velban™), vindestine (Eldesine™), vinorelbine (Navelbine™), paclitaxel (Taxol™)); antibiotic-type agents (e.g., doxorubicin (Rubex™), bleomycin (Blenoxane™), dactinomycin (Cosmegen™), daunorubicin (Cerubidin™), mitomycin (Mutamycin™)); hormonal agents (e.g., aminoglutethimide (Cytadren™); anastrozole (Arimidex™), estramustine (Estracyt™, Emcyt™), goserelin (Zoladex™), hexamethylmelanine (Hexamet™), letrozole (Femara™), anastrozole (Arimidex™), tamoxifen (Estroxyn™, Genox™, Novaldex™, Soltamox™, Tamofen™)); or any combination of any two or more anti-neoplastic agents (e.g., Adriamycin/5-fluorouracil/cyclophosphamide (FAC), cyclophosphamide/methotrexate/5-fluorouracil (CMF)). The antibodies of the invention may also be formulated with compounds and agents, other than those specifically mentioned herein, in accordance with accepted pharmaceutical practice.

In accordance with the mode of administration to be used, and the suitable pharmaceutical excipients, diluents and/or carriers mentioned herein before, compositions of the invention are converted to customary dosage forms such as solutions, orally administrable liquids, injectable liquids, tablets, coated tablets, capsules, pills, granules, suppositories, trans-dermal patches, suspensions, emulsions, sustained release formulations, gels, aerosols, liposomes, powders and immunoliposomes. The dosage form chosen will reflect the mode of administration desired to be used, the disorder to be treated and the nature of the agent to be used. Particularly preferred dosage forms include orally administrable tablets, gels, pills, capsules, semisolids, powders, sustained release formulation, suspensions, elixirs, aerosols, ointments or solutions for topical administration, and injectable liquids.

Skilled persons will readily recognize appropriate dosage forms and formulation methods. The compositions can be prepared by contacting or mixing specific agents and ingredients with one another. Then, if necessary, the product is shaped into the desired formulation. By way of example, certain methods of formulating compositions may be found in references such as Gennaro AR: Remington: The Science and Practice of Pharmacy, 20th ed., Lippincott, Williams & Wilkins, 2000.

The amount of an antibody of the invention in a composition can vary widely depending on the type of composition, size of a unit dosage, kind of carriers, diluents and/or excipients, and other factors well known to those of ordinary skill in the art. The final composition can comprise from 0.0001 percent by weight (% w) to 100% w of the actives of this invention, preferably 0.001% w to 10% w, with the remainder being any other active agents present and/or carrier(s), diluent(s) and/or excipient(s).

Administration of any of the agents or compositions of the invention can be by any means capable of delivering the desired activity (inhibition of tumor cell proliferation) to a target site within the body of a subject. A “target site” may be any site within the body which may have or be susceptible to a proliferative disorder, and may include one or more cells, tissues or a specific tumor.

For example, administration may include parenteral administration routes, systemic administration routes, oral and topical administration. For example, administration may be by way of injection, subcutaneous, intraorbital, ophthalmic, intraspinal, intracistemal, topical, infusion (using e.g. slow release devices or minipumps such as osmotic pumps or skin patches), implant, aerosol, inhalation, scarification, intraperitoneal, intracapsular, intramuscular, intratumoral, intranasal, oral, buccal, transdermal, pulmonary, rectal or vaginal. As will be appreciated, the administration route chosen may be dependent on the position of the target site within the body of a subject, as well as the nature of the agent or composition being used.

The dose of an antibody of the invention or composition administered, the period of administration, and the general administration regime may differ between subjects depending on such variables as the nature of the condition to be treated, severity of symptoms of a subject, the size of any tumor to be treated, the target site to be treated, the mode of administration chosen, and the age, sex and/or general health of a subject. Persons of general skill in the art to which the invention relates will readily appreciate or be able to determine appropriate administration regimes having regard to such factors, without any undue experimentation. Administration of an antibody of the invention is in an amount necessary to at least partly attain a desired response. Administration may include a single daily dose or administration of a number of discrete divided doses as may be appropriate. Administration regimes can combine different modes or routes of administration. For example, intratumoral injection and systemic administration can be combined.

The method may further comprise further steps such as the administration of additional agents or compositions which may be beneficial to a subject having regard to the condition to be treated. For example, other agents of use in treating proliferative disorders (such as the anti-neoplastic agents mentioned above) could be administered. It should be appreciated that such additional agents and compositions may be administered concurrently with the agents and compositions of the invention, or in a sequential manner (for example the additional agents or compositions could be administered before or after administration of the agents or compositions of the invention. It should be appreciated in relation to sequential delivery of agents or compositions, that sequential administration of one agent or composition after the other need not occur immediately, although this may be preferable. There may be a time delay between delivery of the agents or compositions. The period of the delay will depend on factors such as the condition to be treated and the nature of the compositions or agents to be delivered. However, by way of example, the delay period can be between several hours to several days or months.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

We claim:

1. An antibody that specifically binds a trefoil factor 1 (TFF1) polypeptide, wherein said antibody comprises one or both of the following characteristics:
a) said antibody binds to a conformational epitope on said TFF1 polypeptide; and
b) said antibody comprises an antigenic determinant selected from the antigenic determinants shown in Table 4.

2. The antibody of claim 1, wherein said conformational epitope is selected from a conformational epitope shown in Table 3.

3. The antibody of claim 1, wherein said antibody is selected from a multimeric antibody, a chimeric antibody composition comprising a TFF1 binding component and a TFF3 binding component, an antibody produced by a hybridoma cell line selected from 1C6, 3F6, 2C5, 1D, 1A12, 3A2, 3A5, 3B8, 3F4, 3F12, 3G4, 1A11, 2B3, 3B4, 1C4, 2C12, 2A8, 3D7, 1E4, 2E2, 2H4, 1D8 and 2D7, and combinations thereof.

4. An antibody that specifically binds a trefoil factor 1 (TFF1) homodimer or heterodimer polypeptide, wherein said antibody comprises one or both of the following characteristics:
a) said antibody binds to a conformational epitope on said TFF1 polypeptide; and
b) said antibody comprises an antigenic determinant selected from the antigenic determinants shown in Table 2.

5. The antibody of claim 4, wherein said conformational epitope is selected from a conformational epitope shown in Table 1.

6. The antibody of claim 4, wherein said antibody is selected from a multimeric antibody, a chimeric antibody composition comprising a TFF1 binding component and a TFF3 binding component, an antibody produced by a hybridoma cell line selected from 1C6, 3F6, 2C5, 1D, 1A12, 3A2, 3A5, 3B8, 3F4, 3F12, 3G4, 1A11, 2B3, 3B4, 1C4, 2C12, 2A8, 3D7, 1E4, 2E2, 2H4, 1D8 and 2D7, and combinations thereof.

7. An antibody that specifically binds a trefoil factor 3 (TFF3) homodimer, a TFF3 heterodimer polypeptide, or aggregates of the TFF-3 homodimer, wherein said antibody comprises one or both of the following characteristics:
a) said antibody binds to a conformational epitope on said TFF3 polypeptide; and
b) said antibody comprises an antigenic determinant selected from the antigenic determinants shown in Table 6.

8. The antibody of claim 7, wherein said conformational epitope is selected from a conformational epitope shown in Table 5.

9. The antibody of claim 7, wherein said antibody is selected from a multimeric antibody, a chimeric antibody composition comprising a TFF1 binding component and a TFF3 binding component, an antibody produced by a hybridoma cell line selected from 1C6, 3F6, 2C5, 1D, 1A12, 3A2, 3A5, 3B8, 3F4, 3F12, 3G4, 1A11, 2B3, 3B4, 1C4, 2C12, 2A8, 3D7, 1E4, 2E2, 2H4, 1D8 and 2D7, and combinations thereof.

10. A method of treating or preventing cancer, a cell proliferation disorder or a cell survival disorder in a subject in need thereof, comprising administering to said subject an antibody that specifically binds a trefoil factor 1 (TFF1) polypeptide, an antibody that specifically binds a TFF1 homodimer or heterodimer polypeptide, or an antibody that specifically binds a trefoil factor 3 (TFF3) homodimer, a TFF3 heterodimer polypeptide, or aggregates of the TFF-3 homodimer.

11. The method of claim 10, wherein said cancer is an epithelial cancer.

12. The method of claim 11, wherein said epithelial cancer is selected from lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

13. The method of claim 12, wherein said cell proliferation disorder or a cell survival disorder is selected from the group consisting of keratinocyte hyperproliferation, inflammatory cell infiltration, endometriosis, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

14. The method of any claim 10, wherein said subject is a human.

15. The method of claim 14, further comprising the administration of a second compound wherein said second compound is a chemotherapeutic or anti-neoplastic agent.

16. A method of diagnosing a cancer, a cell proliferation disorder or a cell survival disorder in a subject, comprising
a) contacting a test sample from said subject with an antibody that specifically binds a trefoil factor 1 (TFF1) polypeptide, an antibody that specifically binds a TFF1 homodimer or heterodimer polypeptide, or an antibody that specifically binds a trefoil factor 3 (TFF3) homodimer, a TFF3 heterodimer polypeptide, or aggregates of the TFF-3 homodimer; and
b) detecting the level of antibody that binds to said sample,
wherein an increase in the level of antibody binding in said sample compared to the level of binding in a control sample indicates the presence of a cancer, a cell proliferation disorder or a cell survival disorder.

17. The method of claim 16, wherein said cancer is an epithelial cancer.

18. The method of claim 17, wherein said epithelial cancer is selected from lung cancer, colorectal cancer, breast cancer, pancreatic cancer, ovarian cancer, prostate cancer, hepatic carcinoma, gastric carcinoma, endometrial carcinoma, renal carcinoma, thyroid cancer, biliary duct cancer, esophageal cancer, brain cancer, melanoma, multiple myeloma, hematologic tumor, and lymphoid tumor.

19. The method of claim 16, wherein said cell proliferation disorder or a cell survival disorder is selected from the group consisting of keratinocyte hyperproliferation, inflammatory cell infiltration, endometriosis, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

20. The method of claim 16, wherein said subject is a human.
